(19) United States
(12) Patent Application Publication
(10) Pub. No.: US 2025/0255782 A1
(43) Pub. Date: Aug. 14, 2025

(54) COMPOSITIONS FOR USE AS DENTINE SUBSTITUTE

(71) Applicants: Université de Genève, GENEVE (CH); CENTRE NATIONAL DE LA RECHERCHE SCIENTIFIQUE (CNRS), PARIS (FR); SORBONNE UNIVERSITE, PARIS (FR)

(72) Inventors: Tissiana BORTOLOTTO, GENEVE 4 (CH); Ivo KREJCI, GENEVE 4 (CH); Nadine NASSIF, PARIS CEDEX 05 (FR); Miléna LAMA, PARIS CEDEX 05 (FR); Camila BUSSOLA TOVANI, PARIS CEDEX 05 (FR)

(73) Assignees: Université de Genève, GENEVE (CH); CENTRE NATIONAL DE LA RECHERCHE SCIENTIFIQUE (CNRS), PARIS (FR); SORBONNE UNIVERSITE, PARIS (FR)

(21) Appl. No.: 18/849,368

(22) PCT Filed: Mar. 23, 2023
(86) PCT No.: PCT/EP2023/057545

§ 371 Date: Sep. 20, 2024

(30) Foreign Application Priority Data
Mar. 23, 2022 (WO) PCT/IB2022/000166

(51) Int. Cl. A61K 6/75; A61K 6/17; A61K 6/60

(52) U.S. Cl. CPC A61K 6/75; A61K 6/17; A61K 6/60

(57) ABSTRACT

The present disclosure relates to compositions comprising collagen microparticles comprising more than 90% by weight of collagen, biomimetic hydroxyapatite or biomimetic hydroxyapatite precursors and a physiologically compatible aqueous solvent for use in dentine repair.

### FIELD OF THE INVENTION

The present invention relates to the field of repair and regeneration of dentine.

### BACKGROUND OF THE INVENTION

The long-life expectancy of the worlds' population together with the patients' will to keep their teeth all along life has influenced the way how the dental profession deals with the caries process. The traditional surgical model approach for dental care consisting of “carious cavity-restoration-new caries-restoration replacement-tooth weakening-tooth extraction” has been progressively replaced by a much more conservative medical model in which minimally invasive dentistry prevails. In this context, dentists have more and more interest to preserve enamel and dentin as much as possible. Moreover, remineralization of tooth substrate can be considered the foundation for prevention and nonsurgical therapeutic strategies for dental caries.

Dentin is mainly composed of hydroxyapatite (HAp) crystals and type I collagen. Caries disease results from bacteria producing acids that dissolve HAp crystals and disrupt collagen fibers. Unless demineralization process reverses towards mineralization by increasing local pH favoring mineral deposition on dentin, caries process will progress until a tooth cavity is formed. This cavity requires to be filled by different type of materials, and this is the basis of the dentists daily clinical work, to fill cavities which are no more than the late symptoms of a disease that started initially with a simple demineralization from the tooths' surface. Sometimes, the extension and depth of a carious cavity will compromise the tooth's mechanical integrity and pulp (nerve) vitality.

International patent application WO2007/009477 discloses bone repair compositions, comprising a matrix building polymer (i.e. collagen) with inclusions of hydroxyapatite particles (50% of said particles having a size of 5 nm or less) as biomaterial for medical applications such as bone implant material or dental cement. The hydroxyapatite used in WO2007/009477 differs from biomimetic hydroxyapatite at least in terms of chemical composition, size and shape. Also, the compositions are not concentrated enough in collagen so as to obtain a liquid crystal organizations. In addition, WO2007/009477 teaches that collagen may be replaced by gelatin, or that the compositions may be prepared at a temperature of up to 45° C., which thus allows to work above 40° C., the temperature at which collagen is irreversibly denatured to turn into a gelatinized material in vitro. Finally, the compositions exemplified in WO2007/009477 are all dried before use to form a powder, so that collagen turns into a fragile sponge-like material instead of a hydrogel including striated fibrils.

In this context, tooth-like material that is able to “repair” carious dentin is desirable. In particular, there is a need for a tooth-like material that is able to repair demineralized dentin at the late stages of caries disease, that is, when a cavity has been already formed.

### SUMMARY OF THE INVENTION

The invention relates to a composition for use in dentine repair and regeneration comprising:
uncrosslinked and non-denatured collagen microparticles comprising more than 90% by weight of collagen;
biomimetic hydroxyapatite or biomimetic hydroxyapatite precursors; and
a physiologically compatible aqueous solvent.

The invention also relates to a preformed implantable matrix comprising a composition as disclosed herein.

Further aspects of the invention are as disclosed herein and in the claims.

### FIGURES

FIG. 1: TGA thermogram of a hybrid collagen material showing good agreement between initial weights (collagen microparticles contain about 10 wt % water) and measured organic and inorganic contents (initial collagen/hydroxyapatite ratio 1:1).

FIG. 2: DSC analysis of different collagen materials prepared with saline solution displaying similar endothermal peaks typical of collagen denaturation.

FIG. 3: PLM observations of a hybrid collagen solution: bright birefringent textures evidence anisotropic organizations.

FIG. 4: SEM micrograph of a mineralized collagen material displaying partially dissolved collagen microparticles, before fibrillogenesis (left). After fibrillogenesis (right) the material exhibits more defined collagen fibrils.

FIG. 5: SEM micrograph of a mineralized collagen gel (collagen/hydroxyapatite ratio 1:1) displaying fibrillar alignment domains in a dense matrix.

FIG. 6: TEM micrograph of unstained ultrathin section of a collagen/HA 50:50 matrix with high dry matter content displaying co-alignment of collagen fibrils and hydroxyapatite nanoplatelets.

FIG. 7: Energy-dispersive X-ray spectroscopy (EDS) chemical profiles at the interface of the dentin/mixture (in each case: right column: mean C; Middle column: mean P, left column: mean Ca).

FIG. 8: Cross-section scanning electron microscopy (SEM) micro morphology of composition of example 2 on demineralized dentin. The presence of the biomodified layer is marked between the two white lines.

FIG. 9: Cross-section SEM micro morphology of composition of example 3 on demineralized dentin. The presence of the biomodified layer is marked between the two black lines.

FIG. 10: SEM micrograph of the vacuum-dried injectable hybrid material of example 4 displaying the close organic-inorganic integration of mixture 3.

FIG. 11: SEM micrograph of the vacuum-dried injectable hybrid material of example 4 displaying the close organic-inorganic integration of mixture 3.

FIG. 12: Cross-section SEM micro morphology of the vacuum-dried injectable hybrid material of example 4 applied on demineralized dentin. The presence of the bio modified layer is marked between the two white arrows.

FIG. 13: Cross-section SEM micro morphology of the vacuum-dried injectable hybrid material of example 4 showing the biomimetic nature of mix 3 when applied over dentin.

FIG. 14: Cross-section SEM micro morphology of the vacuum-dried injectable hybrid material of example 4 applied on demineralized dentin. The biomimetic interface is marked with the white arrow. See that the biomimetic mixture is very well integrated to the underlying dentin.

### DESCRIPTION OF THE INVENTION

The inventors have discovered that a composition comprising biomimetic hydroxyapatite or amorphous calcium phosphate and dense collagen microparticles is able to repair demineralized dentin. After injection, the composition is biomimetic in terms of microstructure and can serve as a scaffold to promote dentin repair. The major advantage of such composition is that due to its similar composition to dentin, it serves as a neo-substrate for dentin adhesion.

Hence, the present invention relates to a composition as disclosed herein below for use in dentine repair and regeneration, in particular for use in repairing damages to tooth dentin.

### Compositions

The compositions for use in accordance with the present invention comprise:
dense collagen microparticles (i.e. microparticles comprising more than 90 wt % of collagen);
biomimetic hydroxyapatite or biomimetic hydroxyapatite precursors or amorphous calcium phosphate; and
a physiologically compatible aqueous solvent.

The compositions are suitable for injection and/or implantation. The compositions may then be defined as being injectable and/or implantable.

The components of the compositions are as described in details herein below.

### Dense Collagen Microparticles

The term “dense collagen microparticles” as used herein designates collagen microparticles comprising more than 90% by weight of collagen, in particular more than 91%, 92%, 93%, 94%, 95%, 96%, 97%, 98% by weight of collagen, the remaining being water.

The dense collagen microparticles are as disclosed in WO2016/146954.

The dense collagen microparticles are in the form of solid spherical or spheroid particles formed of non-denatured and uncrosslinked collagen. The diameter of the particles typically ranges from 0.05 to 20 μm, in particular from 0.25 to 10 μm, more particularly from 0.4 μm to 3 μm. It is to be understood that the particles diameter ranges refer to the diameter distribution. The particles typically have a diameter ranging from a minimum diameter of 0.05 μm to a maximum diameter of 20 μm.

The term “spheroid” as used herein designates a solid of which the shape assimilates to that of a sphere.

The term “diameter” designates the diameter of the sphere or the greatest diameter of the spheroid. The diameter can be measured for example by electron microscopy or by dynamic light scattering.

The term “non-denatured” as used herein designate a collagen of which the secondary structure of the a-triple helices is preserved. The non-denatured or denatured nature of collagen can be observed for example by calorimetric analysis. Denatured collagen has a calorimetric profile characteristic of a denatured protein (gelatin), with no sign of organized macromolecular domains. Dried collagen (leading to a sponge like material without any striated fibrils) and gelatinized collagen are considered as “denatured” collagen. The use of non-denatured collagen is advantageous in that it will improve the ability of the compositions to behave as a biomimetic scaffold, enable recruitment and activation of hard tissue-forming cells to stimulate dentinogenesis and therefore, regeneration.

The term “uncrosslinked” as used herein designates a collagen in which there are no crosslinking bonds, whether these bonds are the result of chemical, such as treatment by glutaraldehyde, or enzymatical or physical modifications. The absence of crosslinking can be determined for example by electrophoresis.

The dense collagen microparticles may be prepared from a variety of collagen. Hence, the source of collagen is irrelevant. The collagen can be obtained in accordance with the following protocol: a solution of type I collagen is prepared from Wistar rat tail tendons. After excision in a laminar flow cabinet, the tendons are washed in a sterile saline phosphate buffer solution. The tendons are then immersed in a solution of 4 M NaCl in order to remove the remaining intact cells and precipitate some of the proteins of elevated molecular weight. After washing by the saline phosphate buffer solution, the tendons are solubilized in a sterile 500 mM acetic acid solution. The solution obtained is clarified by centrifugation at 41000 g for 2 hours. The proteins other than the collagen are precipitated selectively in an aqueous solution of 300 mM NaCl and removed by centrifugation at 41000 g for 3 hours. The collagen is recovered from the supernatant by precipitation in a solution of 600 mM NaCl followed by centrifugation at 3000 g for 45 minutes. The pellets obtained are solubilized in an aqueous solution of 500 mM acetic acid, then dialysed in the same solvent in order to remove the NaCl ions. The solution is held at 4° C. and centrifuged at 41000 g for 4 hours prior to use. This detailed protocol can be applied to other types of collagen.

The collagen of the dense collagen microparticles has typically a molecular mass ranging from 200 to 450 KDa.

The collagen of the dense collagen microparticles is typically a type I collagen. Nevertheless, the collagen may alternatively be of type II, III, V, XI, XXIV, XXVII, and mixtures thereof.

The dense collagen microparticles may be prepared by a spray-processing technology as disclosed in WO2016/146954. In brief, the spray-processing technology consists in atomizing an acid-soluble collagen solution (non-denatured and uncrosslinked collagen) in order to form a mist of very thin droplets, immediately dried by evaporation of the solvent in a controlled atmosphere (thanks to the high solution/air interface area of the droplets). The concentration of collagen in the acidic collagen solution typically ranges from 0.1 to 10 mg/L. The acidic collagen solution has a pH inferior to 7. The acid is typically acetic acid. The acetic acid concentration in the acidic collagen solution typically ranges from 0.1 to 1000 mM. The atomization is typically performed at a temperature below about 40° C., in particular below about 39° C., 38° C. or 37° C., to obtain a powdered composition. The concentration in the collagen drops is high enough to induce the self-assembly of collagen molecules and a subsequent liquid crystal order, e.g., nematic oriented domains. This strategy allows obtaining within seconds highly concentrated collagen microparticles circumventing the high increase of viscosity of type I collagen solutions that usually prevents fast processing of this protein, and consequently its use at biological concentration.

Therefore, advantageously, the composition comprises 40 mg/mL or of collagen, relative to the total weight of the composition.

### Biomimetic Hydroxyapatite

The chemical formula of biomimetic hydroxyapatite is Ca₁₀₋ₓ(PO₄)₆₋ₓ(CO₃)ₓ(OH)₂₋ₓ with 0≤x≤2. The terms “biomimetic hydroxyapatite” refer to bone-like hydroxyapatite platelets, typically with a length approximately of 10 to 200 nm, and a width from 25 to 100 nm and thickness 1-10 nm, as measured by transmission electron microscopy.

The biomimetic hydroxyapatite is typically in the form of powder.

The biomimetic hydroxyapatite powder may be synthesized following a procedure described by Nassif et al., Chemistry of Materials, 22(12), pp.3653-3663, 2010. Briefly, biomimetic hydroxyapatite is prepared via vapor diffusion of ammonia (NH₃) into an acidic calcium-phosphate (CaCl₂—NaH₂PO₄— or possibly with other salts in particular NaHCO₃) solution based on thermodynamic conditions to avoid the precipitation of other calcium-phosphate phases. For instance, biomimetic hydroxyapatite may be prepared by precipitation of a CaCl₂/NaH₂PO₄ acidic solution (acetic acid, 500 mM) with a calcium-to-phosphate (Ca/P) molar ratio which is consistent with the formation of hydroxyapatite with a formula of Ca₁₀(PO₄)₆(OH₂) or of a CaCl₂/NaH₂PO₄/NaHCO₃ acidic solution (acetic acid, 500 mM) with a calcium-to-phosphate plus carbonate (Ca/[P+C]) molar ratio which is consistent with the formation of hydroxyapatite with a formula of Ca₁₀₋ₓ(PO₄)₆₋ₓ(CO₃)ₓ(OH)₂₋ₓ with 0≤x≤2. The precipitation is triggered by the addition of an ammonia aqueous solution (30%, w/w).

This precipitation method, which is free of any organic additives, has the advantage of being conducted at room temperature within a few hours, without direct pH control, and does not produce any by-product or non-desired (i.e., non-physiological) phases.

It was shown that the synthesis of biomimetic hydroxyapatite as disclosed by Nassif et al., 2010 results in nanoplatelets exhibiting similar self-assembling properties in water as native bone apatites (Wang, Yan, et al. “Water-mediated structuring of bone apatite.” Nature materials 12.12 (2013): 1144-1153). The nanoplatelets have been shown to have a crystalline core and amorphous shell with X-ray diffraction pattern matching that of JCPDS N 9-0432. They typically have an average size of 200×100×5 nm³ and carbonate substitution as observed for bone mineral. Such self-assembling properties are not exhibited by non-biomimetic hydroxyapatite, and in particular with hydroxyapatite particles that do not exhibit an amorphous layer.

It should be noted that the composition of hydroxyapatite can also be modified and in particular enriched with strontium (up to 10% Calcium substitution) to combine anti-osteoporotic effects (Tovani et al. ‘Formation of stable strontium-rich amorphous calcium phosphate: Possible effects on bone mineral’, Acta biomaterialia, 2019). In such case, the strontium-enriched biomimetic hydroxyapatite has typically the following formula: Ca₁₀₋ₓ(PO₄)₆₋ₓSr_{y}(CO₃)ₓ(OH)₂₋ₓ with 0≤x≤2 and 0≤y≤10−x and y being for instance equal to 0.1*(10−x).

### Biomimetic Hydroxyapatite Precursors

The terms “biomimetic hydroxyapatite precursors” refer to the precursor ions leading to the formation of biomimetic hydroxyapatite for instance under conditions described in Nassif et al., Chemistry of Materials, 22(12), pp.3653-3663, 2010.

Suitable biomimetic hydroxyapatite precursors include CaCl₂·2H₂O, NaH₂PO₄ and NaHCO₃ and salts that may be found in the mineral bone composition including salts of magnesium, zinc, fluor and strontium.

The molar ratio Ca/P typically ranges from 1.5 to 2.

The calcium to phosphate plus carbonate ratio (Ca/[P+C]) molar ratio is consistent with the formation of hydroxyapatite (typically 1.67; around 1.2-1.5 for bone tissue) preferably with a formula of Ca₁₀₋ₓ(PO₄)₆₋ₓ(CO₃)ₓ(OH)₂₋ₓ with 0≤x≤2 (Von Euw, scientific reports 2019).

### Amorphous Calcium Phosphate

The terms “amorphous calcium phosphate” refer to amorphous calcium phosphate particles. The amorphous calcium phosphate is typically in the form of powder.

The amorphous calcium phosphate powder may be synthesized by the atomization of the biomimetic hydroxyapatite precursors acidic solution using a spray-processing technology as disclosed in WO2016/146954. The amorphous calcium phosphate powder has a mean size typically ranging from 3 to 6 μm as measured by transmission electron microscopy

### Aqueous Solvent

The aqueous solvent may be any physiologically compatible aqueous solvents. Non limitative examples of suitable aqueous solvents include physiological serum, phosphate buffer, sodium bicarbonate, sterile water, normal saline, blood or blood plasma.

Advantageously, the weight ratio of aqueous solvent to the mixture of dense collagen microparticles and hydroxyapatite or amorphous calcium phosphate typically ranges from 1.8 to 10, preferably from 2 to 9, more preferably from 3 to 8.

When biomimetic hydroxyapatite precursors are used, the weight ratio corresponds to the weight ratio of aqueous solvent to the mixture of dense collagen microparticles and equivalent hydroxyapatite obtained with the biomimetic precursors.

This concentration is advantageous in that the composition is not dry—thus avoiding collagen denaturation—and it is concentrated enough to keep the self-assembly of collagen molecules and subsequent liquid crystal order (with nematic oriented domains), while remaining injectable.

### Optional Therapeutic or Bioactive Agents

The compositions may comprise one or more therapeutic or bioactive agents, such as for example anti-inflammatory agents, saliva, antibiotics, osteogenic proteins, hyaluronic acid and anti-osteoporotic agents (e.g., salts).

The compositions for use in accordance with the present invention typically comprise from 20 mg to 100 mg of dense collagen microparticles per mL of composition, preferably from 40 mg to 80 mg, more preferably from 50 mg to 70 mg.

In some embodiments, the weight ratio of dense collagen microparticles to biomimetic hydroxyapatite or amorphous calcium phosphate ranges from 10:90 to 90:10, preferably 30:70 to 80:20, more preferably 50:50 or 30:70, in the compositions (that may be prepared in accordance with process 1).

The skilled person will readily adjust the weight ratio of dense collagen microparticles to biomimetic hydroxyapatite or amorphous calcium phosphate to adapt the formulation of the compositions to the envisioned use and administration sites.

The compositions of the present invention can be readily implanted or injected or otherwise applied to a site in which there is a need for a dentin repair. For instance, the compositions can be suitably injected with a syringe directly at the site of the defect to be repaired. The compositions have the ability to fill the targeted defect and take the same 3D shape. The compositions are sufficiently adhesive/tacky to hold in place in the defect without external assistance or agents.

Alternatively, the compositions can be injected in a mold to form a preformed matrix. The preformed matrix is implantable. The present invention also relates to a preformed implantable matrix comprising a composition as disclosed herein for use in dentine repair, in particular for use in repairing damages to tooth dentin, more specifically for use in dentine repair when a cavity has been already formed.

The compositions for use in accordance with the present invention may be suitably prepared as disclosed herein below.

When the compositions are prepared in accordance with process 1, the compositions may be more specifically defined as comprising:
dense collagen microparticles (i.e., microparticles comprising more than 90 wt % of collagen);
biomimetic hydroxyapatite platelets or amorphous calcium phosphate; and
a physiologically compatible aqueous solvent.

When the compositions are prepared in accordance with process 2, the compositions may be more specifically defined as comprising:
hybrid dense collagen microparticles (i.e., dense collagen microparticles comprising biomimetic hydroxyapatite precursors); and
a physiologically compatible aqueous solvent that optionally comprise biomimetic hydroxyapatite precursors.

When the compositions are prepared in accordance with process 3, the compositions may be more specifically defined as comprising:
dense collagen microparticles (i.e., microparticles comprising more than 90 wt % of collagen); and
a physiologically compatible aqueous solvent comprising biomimetic hydroxyapatite precursors.

### Processes for Preparing the Compositions

### Process 1: Mixing Dense Collagen Microparticles and Hydroxyapatite or Amorphous Calcium Phosphate

The compositions may be prepared by mixing a desired weight of dense collagen microparticles, typically in the form of powder, with a desired weight of hydroxyapatite or amorphous calcium phosphate powder. The dense collagen microparticles, the hydroxyapatite powder and the amorphous calcium phosphate powder may be prepared as described herein above. The dense collagen microparticles and the hydroxyapatite or amorphous calcium phosphate powder are typically mixed in a mortar. The mixing of the dense collagen microparticles and hydroxyapatite or amorphous calcium phosphate powder is typically made in a weight ratio that is suitably chosen to reproduce the targeted tissue and which can be adapted to the targeted application. Non-limiting examples of suitable dense collagen microparticles to hydroxyapatite or amorphous calcium phosphate powder weight ratio include the following ratios: from 10/90 to 90/10, preferably from 30:70 to 80:20, more preferably 50:50 or 30:70.

After the dense collagen microparticles and the hydroxyapatite or amorphous calcium phosphate powder have been mixed in a suitable weight ratio, an aqueous solvent as described herein above is added to the mixture. The weight ratio of aqueous solvent to the mixture of dense collagen microparticles and hydroxyapatite or amorphous calcium phosphate powder typically ranges from 1.8 to 10 (i.e., in the range from 0.18 mL to 1 mL of solvent per 100 mg of the mixture of dense collagen microparticles and hydroxyapatite or amorphous calcium phosphate powder), preferably from 2 to 9, more preferably from 3 to 8. The mixture may then be supplemented with one or more therapeutic or bioactive agents, such as an anti-inflammatory or anti-osteoporotic agents.

After mixing, the obtained composition, in a paste or liquid form, may be inserted in a sterile syringe.

All steps of the disclosed process are preferably performed in sterile conditions.

The syringe may then be stored in a dry place at a temperature lower than the denaturation temperature of the collagen, preferably in a fridge at 4° C.

Alternatively, the compositions may be prepared by atomizing an acidic solution comprising biomimetic hydroxyapatite precursors and collagen (process 2) or the dense collagen microparticles may be mixed with an aqueous solution containing the biomimetic hydroxyapatite precursors (process 3).

### Process 2: Atomization of Biomimetic Hydroxyapatite Precursors Containing Collagen Solution

The compositions may be prepared by a process comprising the step of atomizing of a solution containing hydroxyapatite precursors and collagen. The solution has typically an acidic pH (i.e. strictly below 7).

The spray-processing technology is performed as disclosed WO2016/146954. The atomization is performed with an acid-soluble collagen solution (non-denatured and uncrosslinked collagen). The concentration of collagen in the acidic collagen solution typically ranges from 0.1 to 10 mg/L. The acidic collagen solution has a pH inferior to 7. The acid is typically acetic acid. The acetic acid concentration in the acidic collagen solution typically ranges from 0.1 to 1000 mM. The collagen solution is mixed with a desired volume/concentration of a biomimetic hydroxyapatite precursors solution (i.e., the acidic collagen solution is supplemented with the ionic precursors of hydroxyapatite). In a preferred set-up, the biomimetic hydroxyapatite precursors solution is made by dissolving biomimetic hydroxyapatite platelets in an acidic solution.

Atomization is typically performed at a temperature below about 40° C., in particular below about 39° C., 38° C. or 37° C., to obtain a non-denatured powdered composition.

The microparticles resulting from the atomization are referred herein as “hybrid dense collagen microparticles”. The hybrid dense collagen microparticles are dense collagen microparticles containing biomimetic ionic precursors (e.g., CaCl₂·2H₂O, NaH₂PO₄ and NaHCO₃). Hybrid microparticles with different ionic compositions may be obtained. Calcium acetate can be used as an alternative to calcium chloride to avoid NaCl precipitation.

The mixing of the hybrid dense collagen microparticles and the physiologically compatible aqueous solvent (containing or not biomimetic hydroxyapatite precursors) is typically made in a weight ratio that is suitably chosen to reproduce the targeted tissue and which can be adapted to the targeted application.

After mixing, the obtained composition, in a paste or liquid form, may be inserted in a sterile syringe.

All steps of the disclosed process are preferably performed in sterile conditions.

The syringe may then be stored in a dry place at a temperature lower than the denaturation temperature of the collagen, preferably in a fridge at 4° C.

### Process 3: Mixing Dense Collagen Microparticles With Biomimetic Hydroxyapatite Precursors Solution

The compositions may be prepared by mixing a desired weight of dense collagen microparticles, typically in the form of powder, with a desired volume of a biomimetic hydroxyapatite precursors solution. The dense collagen microparticles and the biomimetic hydroxyapatite precursors solution may be prepared as described herein above. The dense collagen microparticles and the biomimetic hydroxyapatite precursors solution are typically mixed in a mortar. The mixing of the dense collagen microparticles and the biomimetic hydroxyapatite precursors solution is typically made in a weight ratio that is suitably chosen to reproduce the targeted tissue and which can be adapted to the targeted application. The volume of biomimetic hydroxyapatite precursors solution added to the dense collagen microparticles typically leads to a final concentration of 80 mg/ml of collagen.

After mixing, the obtained composition, in a paste or liquid form, may be inserted in a sterile syringe.

All steps of the disclosed process are preferably performed in sterile conditions. The syringe may then be stored in a dry place at a temperature lower than the denaturation temperature of the collagen, preferably in a fridge at 4° C.

Embodiments of the present invention will now be described by way of the following examples which are provided for illustrative purposes only, and not intended to limit the scope of the disclosure.

### EXAMPLES

Example 1: Injectable Hybrid Material (Collagen/Hydroxyapatite Ratio 50:50) in 0.9% Saline

### Synthesis of Carbonated Hydroxyapatite

The synthesis of carbonated hydroxyapatite was performed in accordance with the procedure described by Nassif et al. (Chemistry of Materials, 22(12), pp.3653-3663, 2010).

A solution of 110 mM CaCl₂·2H₂O, 33 mM NaH₂PO₄ and 33 mM NaHCO₃ was prepared in 500 mM acetic acid. The pH was adjusted to 2.2 with HCl solution at 37%. Two flasks (35 mL) were filled with 20 mL of this solution and placed in a hermetically sealed chamber (i.e., put in a 1 L beaker covered with paraffin), in the presence of a third vial containing 8 mL of an aqueous solution of NH3 28-30% by mass. Before closing, these 3 flasks were covered with parafilm pierced with 6 holes using a needle in order to slow down the gaseous diffusion of the ammonia. The device was then left for 6 days. Then, the precipitate was collected by centrifugation at room temperature (20 minutes at 6000 rpm), washed with ultrapure water until the pH of the supernatant is close to that of the washing water. The white powder obtained was finally dried in an oven at 37° C. for 7 days. The dry powder was then finely milled in a mortar with a pestle to obtain a fine powder.

### Synthesis of Collagen Microparticles by Aerosol

The synthesis of collagen microparticles was performed in accordance with the procedure described by Nassif et al. (Paris, 2018. Injectable collagen suspensions, the preparation method thereof, and the uses thereof, particularly for forming dense collagen matrices. U.S. patent application Ser. No. 15/558,787) and Lama et al. (Self-Assembled Collagen Microparticles by Aerosol as a Versatile Platform for Injectable Anisotropic Materials. Small, p. 1902224, 2019).

A collagen solution concentrated to 1.2 mg/mL was obtained by diluting a collagen stock solution (usually 1.3 to 5 mg/mL) in acetic acid (500 mM). 250 ml of said solution was dried in a spray-dryer (Büchi B290). The spray-dryer was placed under a fume hood next to a mobile reversible air conditioner. The temperature under the fume hood should ideally be maintained between 19° C. and 21° C. (unfavorably above 25° C.). The injection speed of the collagen solution (at 1.2 mg/mL) was controlled by the peristaltic pump of the atomizer and was equal to 0.6 mL/min. The set temperature of the nozzle is maintained at 30° C. The actual temperature of the nozzle oscillates between 34° C. and 35° C. after one hour of stabilization at vacuum (before starting the peristaltic pump). The internal temperature of the system, measured between the drying column and the particle collection cyclone, is between 19° C. and 25° C. The air flow responsible for droplet shearing at the nozzle outlet is 414 L/h. The suction power, which controls the drying of the droplets between the nozzle outlet and the collector, is set at 50% of the maximum capacity of the drying system, i.e., 20 m3/h. The “nozzle” parameter, which is used to prevent coagulation of the solution at the end of the nozzle, is set at 2. Aluminum is placed on both sides of the joint between the column and the cyclone to avoid heat loss as much as possible. The formed particles are collected by a high-performance cyclone connected to a flask. In order to recover all the powder remaining on the walls of the cyclone and to maximize the yield, the temperature set point is turned off at the end of the atomization and the suction is increased in 10% steps, from 50% (20 m3/h) to 100% (40 m3/h) by waiting 5 minutes per step. The process efficiency is between 50% and 60%. To ensure sterile conditions, a commercial device of filters of different sizes sold by BEKO technologies can be used. It is also recommended to sterilize the whole setup with >94° ethanol before spraying the collagen.

### Preparation of the Injectable Composition

60 mg of the collagen powder obtained as disclosed herein above and 60 mg of hydroxyapatite powder obtained as disclosed herein were mixed in a mortar. 1 mL of sterile saline (NaCl 0.9%) was added in the mortar. The whole was mixed for about one minute to obtain a homogeneous paste. The paste was transferred into an empty 1 ml syringe. The plunger was put back in place. The paste was then ready to be injected into the defect.

### Preparation of the Pre-Formed Matrices

The above protocol is repeated. The mixture is injected through the syringe into a silicone mold of the desired dimensions and total volume of 1 mL. Fibrillogenesis (gelation) is performed under ammonia vapor overnight. The gel is then removed from the mold and rinsed with saline to until reaching neutral pH. The material can then be implanted in a cavity corresponding to the shape of the mold.

### Characterization of the Injectable and Pre-Formed Materials

### Methods:

Thermogravimetric analysis (TGA): Experiments were performed with a NIETZSCH STA 409PC instrument on a thermo-microbalance under an oxidizing atmosphere from room temperature to 850° C. with a heating rate of 5° C./min.

Differential scanning calorimetry (DSC): Experiments were performed with a TA Q-20 machine. The heating rate was set at 5° C./min and the temperature range from 20° C. to 80° C. About 20 mg piece of material was weighed and placed in a sealed aluminum pan. An empty sealed aluminum pan was used a reference.

Polarized light microscopy (PLM): The materials were placed without any treatment between a glass slide and a coverslip. Observations were made using a transmission Zeiss Axiolmager A2 POL. The microscope is equipped with the standard accessories for examination of birefringent samples under polarized light (i.e., crossed polarizers) and an AxioCam CCD camera.

Scanning electron microscopy (SEM): Samples were fixed in 2.5% glutaraldehyde solution. After washing in cacodylate/saccharose buffer solution, they were dehydrated through ethanol baths (from 30% to 100% ethanol). Supercritical CO2 drying was performed by a CPD-300 (Leica). Dried samples were cut into pieces, put on carbon tape covering sample holders, covered with 15 nm gold layer. Observations were carried out by using a Hitachi S-3400N microscope operating at 3 kV and 30 pA.

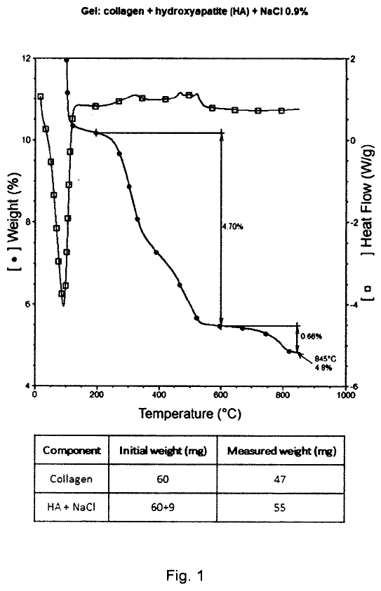

The final composition of the materials is consistent with that of initial mixture, taking into account the presence of water in the collagen microparticles (about 10%) (FIG. 1).

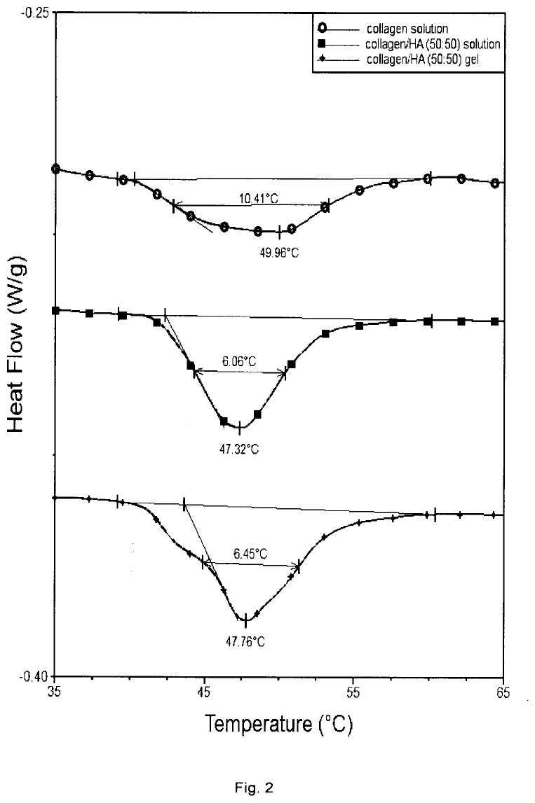

The denaturation temperature of collagen is about 48° C. This is close to the denaturation temperature reported for collagen gels (Tiktopulo and Kajava, 1998) indicating that the addition of saline can promote fibrillogenesis. Indeed, the denaturation temperature remains unchanged when fibrillogenesis is induced by ammonia vapors (mineralized collagen gel). The addition of hydroxyapatite to the collagen microparticles and saline mixture seems to induce favorable interactions: the denaturation enthalpy is higher and the width at mid-height of the endotherm is less important (FIG. 2). This means that the addition of HA would tend to homogenize the collagen fibril (or microfibril) population.

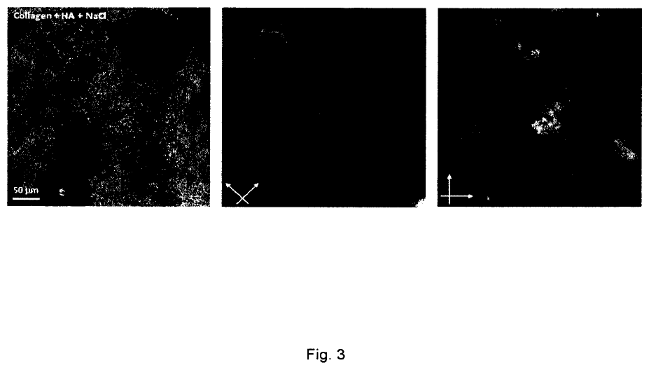

As observed by PLM (FIG. 3), the solution shows domains of birefringence testifying to the anisotropy of the material, and confirming that the addition of hydroxyapatite under these conditions does not prevent the self-assembly of collagen in liquid crystal phases.

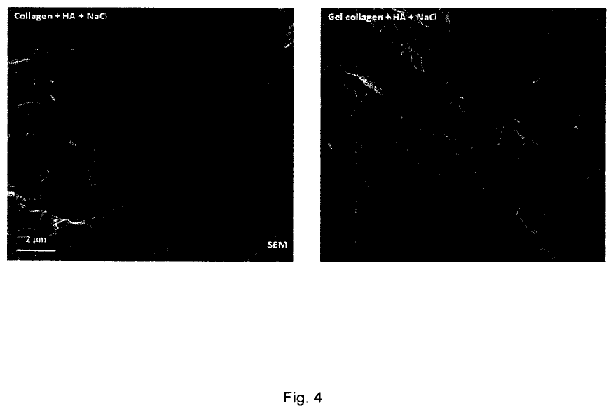

This local anisotropy can be seen by SEM through the observation of aligned mineralized collagen fibril groups (FIG. 4). Before fibrillogenesis, the material also shows partially dissolved collagen microparticles. The dissolution of the microparticles can be modulated by the mixing time before injection. After fibrillogenesis, more defined fibrils are observed.

### Example 2: Injectable Hybrid Material (Collagen/Hydroxyapatite Ratio 50:50) in Acetic Acid 2 mM

### Preparation of the Injectable Hybrid Material

40 mg of the collagen powder obtained as disclosed herein above and 40 mg of the hydroxyapatite powder obtained as disclosed herein above are mixed in a mortar. 0.15 mL of 2 mM acetic acid is added to the mortar. The whole is mixed for about one minute to obtain a homogeneous paste. The paste was transferred into an empty 1 mL syringe. The plunger was put back in place. The paste was then ready to be injected into the defect.

### Characterization of the Material

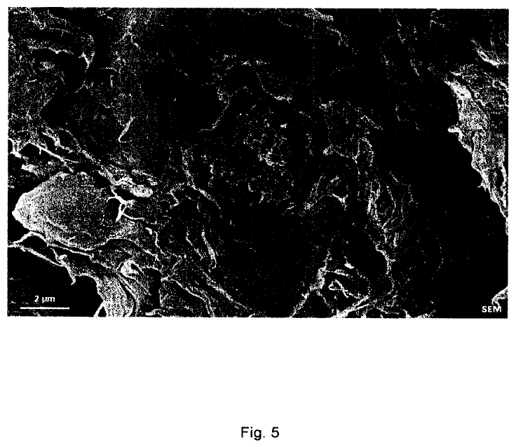

SEM observation shows fibrillar alignment domains (FIG. 5). The material appears dense.

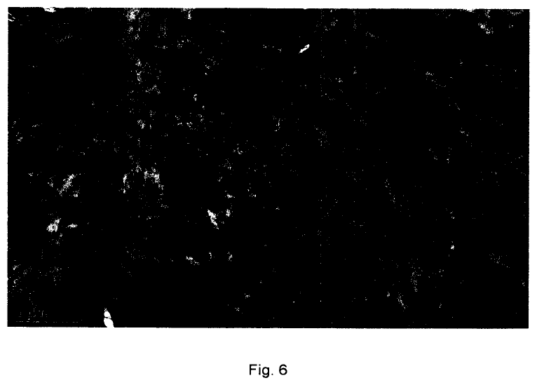

SEM reveals areas of co-alignment of collagen fibrils and hydroxyapatite nanoplatelets, resembling those observed in compact bone (FIG. 6).

### Example 3: Injectable Hybrid Material (Collagen/Hydroxyapatite Ratio 30:70) in Acetic Acid 2 mM

### Preparation of the Injectable Hybrid Material

24 mg of the collagen powder obtained as disclosed herein above and 56 mg of the hydroxyapatite powder obtained as disclosed herein above are mixed in a mortar. 0.15 mL of 2 mM acetic acid is added to the mortar. The whole is mixed for about one minute to obtain a homogeneous paste. The paste was transferred into an empty 1 ml syringe. The plunger was put back in place. The paste was then ready to be injected into the defect.

### Example 4: Injectable Hybrid Material (Collagen/Amorphous Calcium Phosphate Ratio 30:70) in Acetic Acid 2 mM

### Preparation of Amorphous Calcium Phosphate

The amorphous calcium phosphate powder is synthesized by atomization of a biomimetic hydroxyapatite precursors acidic solution of 110 mM CaCl₂·2H₂O, 33 mM NaH₂PO₄ and 33 mM NaHCO₃ in 500 mM acetic acid using a spray-processing technology as disclosed in WO2016/146954.

### Preparation of the Pre-Formed Hybrid Material

40 mg of the collagen powder obtained as disclosed herein above and 40 mg of the amorphous calcium phosphate powder obtained as disclosed herein above are mixed in a mortar. 0.15 mL of 2 mM acetic acid is added to the mortar. The whole is mixed for about one minute to obtain a homogeneous paste. The paste can be injected via a 1 ml syringe into a mold or spread into a mold with a spatula. Fibrillogenesis is performed under ammonia vapors for three hours. The gel is then demolded and rinsed with PBS until reaching neutral pH. The material can then be implanted in a cavity corresponding to the shape of the mold.

### Example 5: Injectable and pre-Formable Material (Dense Collagen Microparticles Mixed With Biomimetic Hydroxyapatite Precursors Solution)

### Preparation of the Injectable Composition

90 mg of the collagen powder obtained as disclosed herein above was mixed with 1 ml of biomimetic hydroxyapatite precursors solution obtained as disclosed herein. The whole was mixed for about one minute to obtain a homogeneous paste. The paste was transferred into an empty 1 ml syringe. The plunger was put back in place. The paste was then ready to be injected into the defect.

### Preparation of the Pre-Formed Matrix

The above protocol is repeated. The mixture is injected through the syringe into a silicone mold of the desired dimensions and total volume of 1 mL. Fibrillogenesis (gelation) is performed under ammonia vapor overnight. The gel is then removed from the mold and rinsed with saline to until reaching neutral pH. The material can then be implanted in a cavity corresponding to the shape of the mold.

### Example 6: Synthesis of Hybrid Collagen Microparticles by Aerosol

The synthesis of collagen microparticles was performed in accordance with the procedure described by Nassif et al. (Paris, 2018. Injectable collagen suspensions, the preparation method thereof, and the uses thereof, particularly for forming dense collagen matrices. U.S. patent application Ser. No. 15/558,787) and Lama et al. (Self-Assembled Collagen Microparticles by Aerosol as a Versatile Platform for Injectable Anisotropic Materials. Small, p.1902224, 2019). In addition, the salts present in biomimetic hydroxyapatite precursor were added to the low concentration collagen acidic collagen solution before the atomization leading to the final composition: 2 mg/mL collagen, 500 mM acetic acid, 110 mM CaCl₂·2H₂O, 33 mM NaH₂PO₄ and 33 mM NaHCO₃. The composition of ionic precursors can be modified to form hybrid collagen microparticles with different mineral/collagen ratios, and loaded with different therapeutic ions e.g., Sr²⁺, Mg²⁺, Zn²⁺.

For example, SrCl₂·6H₂O may be added to the biomimetic hydroxyapatite precursors solution to obtain a 10% Sr²⁺ in relation to Ca²⁺ (mol/mol).

### Preparation of the Injectable Composition

90 mg of the collagen powder obtained as disclosed herein above was mixed with 1 mL of 500 mM acetic acid. The whole was mixed for about one minute to obtain a homogeneous paste. The paste was transferred into an empty 1 mL syringe. The plunger was put back in place. The paste was then ready to be injected into the defect. Different weight of ionic may be used to obtain different mineral/collagen ratios.

### Preparation of the Pre-Formed Matrix

The above protocol is repeated. The mixture is injected through the syringe into a silicone mold of the desired dimensions and total volume of 1 mL. Fibrillogenesis (gelation) is performed under ammonia vapor overnight. The gel is then removed from the mold and rinsed with saline to until reaching neutral pH. The material can then be implanted in a cavity corresponding to the shape of the mold.

### Example 7: Study of Ca and P Ions Restitution to Demineralized Deep Dentin From Class I Cavities by Compositions of Examples 2 and 3

### Preparation of Tooth Cavities

Deep class I cavities were prepared in extracted molars that were anonymously collected. Intratubular fluid flow with a solution of phosphate buffer saline (PBS) and horse serum was performed in order to simulate the biological environment of a living tooth as disclosed in Bortolotto T, Onisor I, Krejci I. Proximal direct composite restorations and chairside CAD/CAM inlays: Marginal adaptation of a two-step self-etch adhesive with and without selective enamel conditioning. Clin Oral Invest 2007; 11:35-43.

After cavity preparation four dentin treatments were considered.

### Positive Control on Sound Dentin

The as-prepared tooth cavity located on sound dentin was filled with a glass ionomer cement (Fuji IX, GC) and stored for 15 days at 37° C. under dentinal perfusion.

### Negative Control on Demineralized Dentin

The as-prepared tooth cavity was etched with a 37% H₃PO₄ aqueous solution for 20 seconds then filled with a glass ionomer cement and stored for 15 days at 37° C. under dentinal perfusion.

### Preparation of Demineralized Dentin Coated With Composition of Example 2 or 3

The as-prepared tooth cavity was etched with a 37% H₃PO₄ aqueous solution for 20 seconds. Then a layer of compositions of example 2 or example 3 was applied. A thin layer of light cured bonding agent (bond of Optibond FL) was placed on top in order to isolate the mixture layer from the cement and avoid any chemical interaction. After applying the glass ionomer cement, teeth were stored for 15 days at 37° C. under dentinal perfusion.

### Characterization of the materials

The tooth samples were sectioned, polished and scanning electron microscopy (SEM) micromorphological assessment and energy dispersive spectroscopy (EDS) chemical profiles at the interface dentin/mixture were extracted.

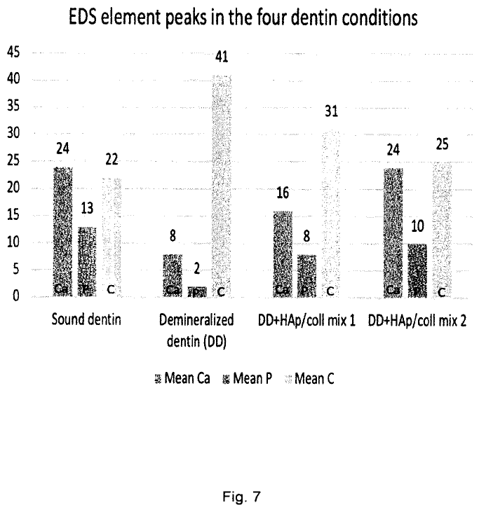

As assessed by EDS, Calcium (Ca), Phosphorous (P) and Carbon (C) peaks varied according to dentin surface treatment types. C peaks were associated with the amount of exposed collagen as peaks up to 41 were observed in demineralized dentin (DD) when peaks of Ca (8) and P (2) were the lowest (FIG. 7).

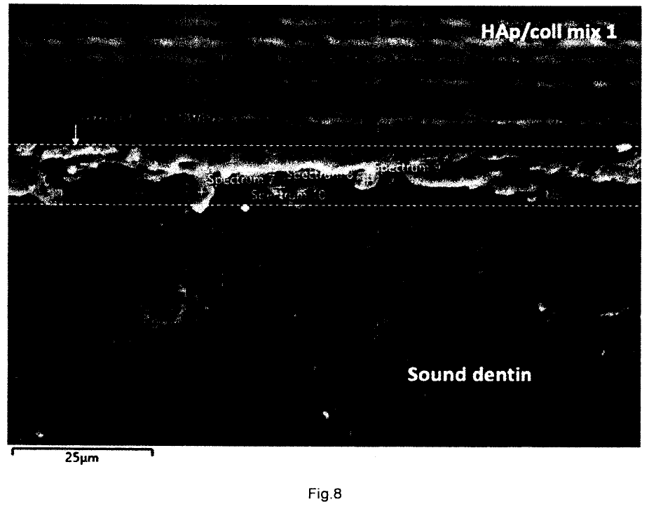

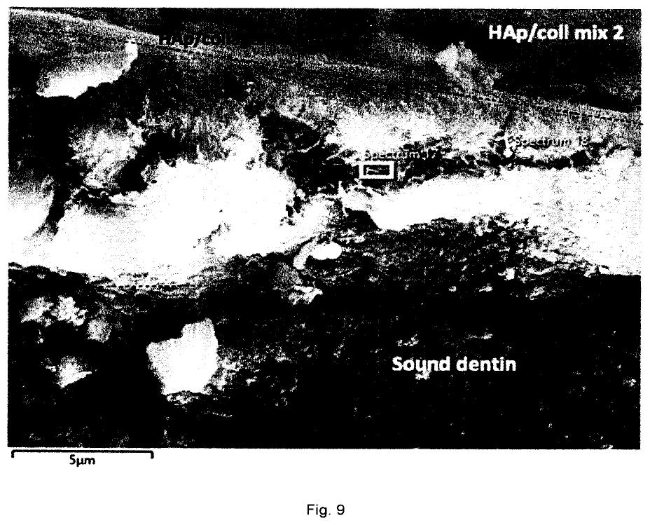

Compositions of examples 2 (HAp coll mix 1) and 3 (HAp coll mix 2) presented the closest peaks to sound dentin, indicating that biomodification occurred on demineralized dentin surface. SEM micrographs confirmed these assumptions, in view of the morphology of a layer that was well integrated on dentin surface (FIGS. 8 and 9).

Therefore, it appears that recovery of demineralized dentin was possible by the integration of a newly-formed hydroxyapatite/collagen layer with similar characteristics as the one observed on sound dentin, especially with composition of example 3 (collagen/hydroxyapatite ratio of 30:70; “HAp coll mix 2”).

### Example 8: Study of Ca and P Ions Mapping of Demineralized Deep Dentin From Class I Cavities With Composition of Example 4 (Amorphous Calcium Phosphate 40 mg+Collagen/Amorphous Calcium Phosphate 44,4 mg Ratio 70/30 in Acetic Acid 2 mM: Mix 3)

The following steps were identical to Example 7: preparation of tooth cavities, positive control on sound dentin, negative control on demineralized dentin, preparation of demineralized dentin coated with compositions of example 4 and characterization of the materials.

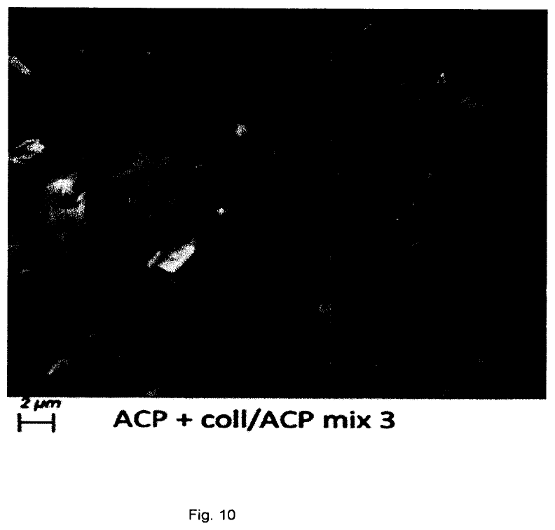

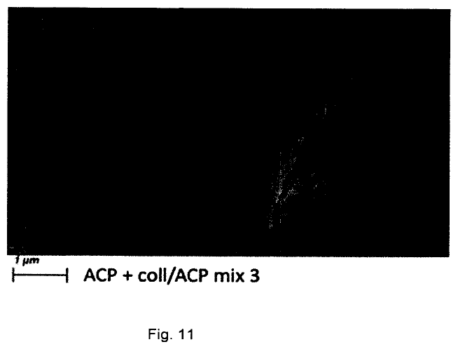

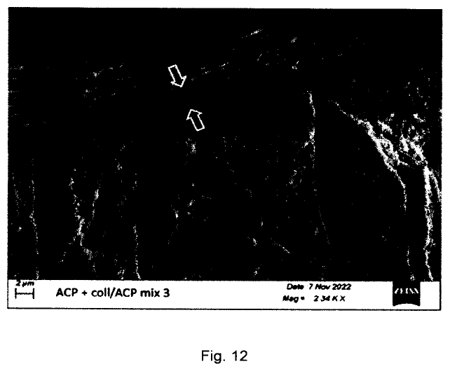

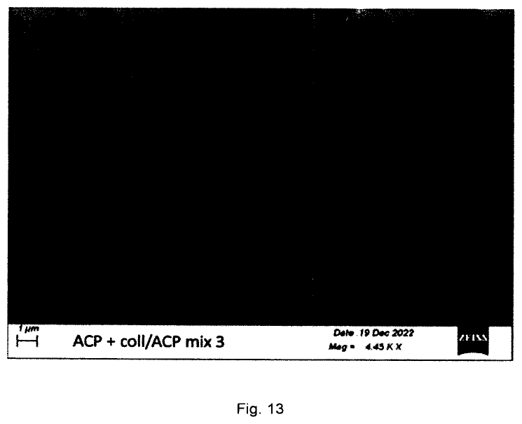

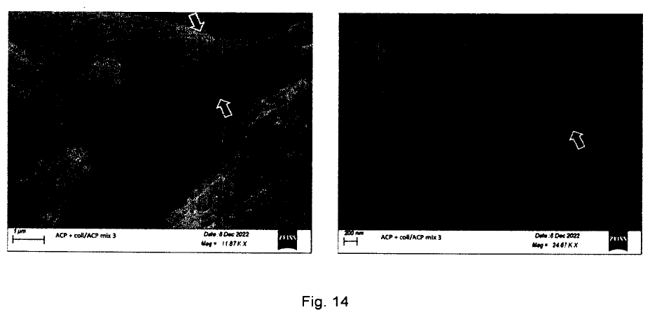

SEM micrographs showed a mineralized collagen material displaying a very tight integration between both components (FIGS. 10 and 11). The interfacial characteristics between dentin and mix 3 were typical of a biomimetic neo layer with a very similar structure to underlying dentin (FIGS. 12-14).

1. A method for repairing and regenerating dentin comprising implanting or injecting or applying an effective amount to a site in which there is a need for a dentin repair an effective amount of a composition comprising:
uncrosslinked and non-denatured collagen microparticles comprising more than 90% by weight of collagen;
biomimetic hydroxyapatite or biomimetic hydroxyapatite precursors or amorphous calcium phosphate; and
a physiologically compatible aqueous solvent.

2. The method of claim 1, wherein the collagen microparticles have a diameter ranging from 0.05 to 20 μm as measured by electron microscopy.

3. The method of claim 1, wherein the collagen microparticles are type I collagen microparticles.

4. The method of claim 1, wherein the physiologically compatible aqueous solvent is physiological serum, phosphate buffer, sodium bicarbonate or blood.

5. The method of claim 1, wherein the composition further comprises one or more therapeutic or bioactive agents.

6. The method of claim 1, wherein the collagen microparticles to biomimetic hydroxyapatite weight ratio or the collagen microparticles to biomimetic hydroxyapatite precursors weight ratio ranges from 10:90 to 90:10.

7. The method of claim 1, wherein the weight ratio of aqueous solvent to the mixture of dense collagen microparticles and hydroxyapatite or amorphous calcium phosphate typically ranges from 1.8 to 10.

8. The method of claim 1, for repairing damages to tooth dentin.

9. The method of claim 8, when a cavity has been already formed.

10. The method of claim 6, wherein the collagen microparticles to biomimetic hydroxyapatite weight ratio or the collagen microparticles to biomimetic hydroxyapatite precursors weight ratio ranges from 30:70 to 80:20.

11. The method of claim 6, wherein the collagen microparticles to biomimetic hydroxyapatite weight ratio or the collagen microparticles to biomimetic hydroxyapatite precursors weight ratio is 30:70.

12. The method of claim 7, wherein the weight ratio of aqueous solvent to the mixture of dense collagen microparticles and hydroxyapatite or amorphous calcium phosphate typically ranges from 2 to 9.

13. The method of claim 7, wherein the weight ratio of aqueous solvent to the mixture of dense collagen microparticles and hydroxyapatite or amorphous calcium phosphate typically ranges from 3 to 8.
